(19) United States
(12) Patent Application Publication
(10) Pub. No.: US 2009/0024089 A1
(43) Pub. Date: Jan. 22, 2009

(54) LONG TAPERED DILATOR

(76) Inventors: JONATHAN A. LEVINE, Clarendon Hills, IL (US); JEROME PRIEST, North Grosvenordale, CT (US)

Correspondence Address: BLANK ROME LLP, ONE LOGAN SQUARE, PHILADELPHIA, PA, 19103, US

(21) Appl. No.: 12/109,978

(22) Filed: Apr. 25, 2008

Related U.S. Application Data
(60) Provisional application No. 60/913,878, filed on Apr. 25, 2007.

(51) Int. Cl. A61M 29/00

(52) U.S. Cl. 604/104; 606/191; 29/428

(57) ABSTRACT

A dilator assembly used for the dilatation of vessel, tracts, or cavities. The dilator assembly comprises a hub portion, a shaft portion, and a long tapered tip portion having a taper no greater than 4 Fr per 1 cm. In certain aspects, the taper of the tapered tip portion is between approximately 1 Fr per 1 cm to 1 Fr per 1.5 cm. The present invention obviates the need for pre-dilatation. The dilator shaft portion and/or the distal tapered tip portion are flexible, providing excellent tractability, being capable of bending along a guide wire with minimal physical force and without kinking, to tract the curvature of a vessel, tract, or cavity. Additionally, a section of the shaft portion can be is capable of similarly bending along a guide wire. The present invention functions as a dilator, and tracts like a catheter over a guide wire.

### RELATED INVENTION

This application claims benefit of U.S. Provisional Application Ser. No. 60/913,878, filed Apr. 25, 2007, entitled “Long Tapered Dilator,” which is incorporated herein by reference.

### FIELD OF THE INVENTION

This invention relates to invasive medical devices which aid in the dilatation of human blood vessels, tracts, and cavities. In particular, this invention relates to a dilator with a flexible long tapered tip which can be used alone or with a sheath.

### BACKGROUND OF THE INVENTION

Catheterization is widely used in modern medical procedures. Catheters used for the introduction or removal of fluids may be located in various vascular locations and cavities throughout the body of a patient, and can be used for introduction of fluids to the body or removal of fluids from the body. Catheters can also be used to introduce medical devices, such as stents, to a desired vascular location and/or cavity.

The current practice of percutaneously accessing veins and arteries for both diagnostic and therapeutic procedures involves several widely employed and accepted steps. First, a desired vessel is punctured with a hollow bore needle attached to a syringe. Blood return in the syringe by aspiration confirms the intravascular location of the needle tip. A guidewire is subsequently advanced through the needle into the vessel lumen, and the needle is removed. The resulting initial opening is of relatively small size, about 4˜5 French size (Fr). Next, the diameter of the access tract is dilated over the guidewire using sequentially larger individual dilators (pre-dilatation). Depending on the ultimate size required and other factors, such as the size of the patient and tissue resistance, this process can involve anywhere from one to several different sized dilators and dilatations. When the desired access tract size is achieved, the dilator is removed over the guidewire. In some instances, the catheter can then be directly advanced over the guidewire into the dilated access tract. More commonly, though, a sheath is used in combination with the dilator. The sheath/dilator assembly is advanced over the guidewire to the desired depth. The dilator is then removed over the guidewire. The sheath is left in the vessel to serve as a stable and protective conduit through which subsequent catheters and balloons can be passed into the vessel without the blood loss, vessel trauma and pain that would occur if such devices were introduced and withdrawn without the sheath in place. The sheath is generally removed from the vessel after the procedure.

Multiple dilators of increasing size are often necessary for the pre-dilatation step, because the diameter of the sheath is often several French sizes larger than the needle used to access the vessel. Attempts to advance the vascular sheath over the guidewire, without first dilating the access tract, would likely result in vessel and guidewire damage, with subsequent loss of the access and/or the possibility of severe bleeding. Typically, one to three dilators are used, depending on the size of the sheath intended for insertion. This practice, while effective, has a number of disadvantages; some with serious potential consequence. For instance, there is repeated trauma to the vessel. During arterial interventions, adequate hemostasis must be achieved during sequential dilator exchanges to prevent unnecessary blood loss, hematoma, or pseudoaneurysm formation. This results in added difficulty for the operating physician. For example, if a 10 Fr sheath is used, enough pressure must be applied to control not only the original 5 Fr arterial hole, but then potentially the 6, 8 and 10 Fr holes, until the sheath is in place. Every time an operating physician must withdraw a dilator over a wire to exchange it for a second, third, or fourth dilator, the physician runs the risk of two very serious consequences: losing access or kinking the guidewire. Also, exchanging dilators is often results in considerable patient discomfort.

Current dilators are rigid, having a straight shaft with short, tapered distal end. The taper of current dilators is too abrupt to allow passage of larger diameter sheaths without first dilating the access tract with separate sequential dilators. Because of their extremely poor taper and stiffness, current larger dilators have the potential to kink the guidewire, resulting in the potential loss of access. Even after what is considered sufficient pre-dilatation, sheath insertion can be difficult because of the extremely short taper of the sheath's introducer. This is especially common with heavily calcified vessels, larger patients, and those with heavily scarred groins from previous bypass or orthopedic surgery. Use of multiple dilators also increases procedure cost.

Commercially available tunneled hemodialysis catheter kits usually come with several dilators. For example, a 14.5 Fr catheter typically has two dilators of increasing size, in addition to a tear-a-way sheath/dilator assembly. Larger catheters kits, typically 16/17 Fr, have three dilators of increasing size, plus a tear-a-way sheath/dilator assembly. Micro-puncture sets create an initial vessel opening of only 4 or 5 Fr. The operating physician's options are to either go straight from the four or five Fr micro-puncture set to the 14˜17 Fr catheter, which, as discussed above, is very dangerous, or to use multiple dilators which costs money and is fraught with the above mentioned problems. Few physicians will go from the micro-puncture set to the existing dilator/tear-a-way sheath assembly without any pre-dilating. There exists a need for a better dilator system to maximize the efficiency of larger diameter sheath and catheter placement, while minimizing potential risk to the patient, blood loss, as well as procedure cost.

### SUMMARY OF THE INVENTION

The present invention is a dilator assembly for the dilatation of vessel, tract, or cavity, comprising a dilator, having a proximal hub portion, a shaft portion, and a flexible distal tapered tip portion. The exterior surface of the shaft portion smoothly transitions to the exterior surface of the flexible distal tapered tip portion. The hub portion, the shaft portion, and the flexible distal tapered tip portion have a continuous hollow center that forms a guidewire passage.

In one aspect, the outer diameter of the distal end of the flexible distal tapered tip portion is about French size 4 to French size 5. In another, the outer diameter of the shaft portion is about French size 8 to French size 30. Generally, the flexible distal tapered tip portion has a taper of no greater than 4 Fr per 1 cm. In some embodiments, the flexible distal tapered tip portion is tapered between approximately 4 Fr per 1 cm and 1 Fr per 2 cm. In other embodiments, the flexible distal tapered tip portion is tapered between approximately 2 Fr per 1 cm and 1 Fr per 2 cm; or between approximately 1 Fr per 1 cm and 1 Fr per 1.5 cm.

The flexible distal tapered tip portion and the shaft portion can be made of a flexible thermoplastic polymer, can be made of Nylon 12, and can be made by an extrusion process.

In certain aspects of the invention, an exterior surface of the flexible distal tapered tip portion and/or the shaft portion have at least one marking indicating the outer diameter of the flexible distal tapered tip portion at one or more points along a longitudinal length of the flexible distal tapered tip portion. In other aspects, the exterior surface of the flexible distal tapered tip portion and/or the shaft portion have at least one marking indicating a depth (or length) from a distal end of the tapered tip portion to the at least one marking.

The present invention can further comprise a sheath having a tubular portion and a sheath hub portion. The sheath may be a tear-away sheath, and/or could comprise a hemostasis valve within a sheath hub portion, and a side port. The dilator could further comprise a coupling means at a distal end of the dilator hub portion that couples with the sheath hub portion. The tubular portion of the sheath has an exterior surface that could have at least one marking indicating a depth (or length) from a distal end of the tubular portion to the at least one marking.

The present invention also provides a method of making a tapered tip dilator including the steps of continuously forming a shaft portion and a tapered tip portion with a hollow center channel, using a variable speed extrusion process, separating an individual section of a shaft portion and a tapered tip portion, wherein the center channel extends to a proximal opening at a proximal end of the shaft portion and to a distal opening at a distal end of the tapered tip portion, and shaping the distal tip opening of the tapered tip portion. Shaping the distal tip opening can include the steps of heating a section of the dilator long tapered tip portion, and compressing the long tapered tip portion in a die.

The method of making a dilator tapered tip portion can include the steps of forming a dilator hub having a distal end, a proximal end, and a hollow center channel, and connecting a distal end of the dilator hub to a proximal end of the shaft portion, whereby the dilator hub, the shaft portion, and the flexible distal tapered portion have a continuous hollow center that forms a guidewire passage.

### BRIEF DESCRIPTION OF THE DRAWINGS

The present invention will be better understood with reference to the following description taken in combination with the drawings. For the purpose of illustration, there are shown in the drawings certain embodiments of the present invention. In the drawings, like numerals indicate like elements throughout. It should be understood, however, that the invention is not limited to the precise arrangements, dimensions, and instruments shown. In the drawings:

FIG. 1 is a perspective view of one embodiment of the present invention dilator;

FIG. 2 is a side view of a representative example of an existing dilator tip;

FIG. 3A is a side view of an expanded long tapered tip portion of one embodiment of the present invention dilator;

FIG. 3B is a cross section taken along line 3B-3B of FIG. 3A;

FIG. 3C is a cross section taken along line 3C-3C of FIG. 3A;

FIG. 4 is a side view of a catheter introducer comprising an embodiment of the present invention dilator and a tear-away sheath; and

FIG. 5 is a side view of a catheter introducer comprising an embodiment of the present invention dilator and a sheath with a hemostasis valve and a side port.

### DETAILED DESCRIPTION OF THE INVENTION

The words “proximal” and “distal” refer to directions away from and closer to, respectively, the insertion tip of a dilator to the present invention. The outer diameter of the dilator is expressed in terms of French size (Fr), which is defined as 1 Fr=⅓ mm.

A dilator generally has a long shaft portion and a proximal hub. The shaft portion of the dilator usually has a tapered tip on its distal end. The dilator also has a hollow center, which runs along the entire length of the dilator. The center channel forms a passage for the guidewire to pass through the center of the dilator.

A sheath has a long thin walled tubular portion and a sheath hub. The tubular portion of the sheath has a slightly tapered distal end and an internal diameter slightly larger than the outer diameter of the dilator. The sheath fits outside of the dilator and forms a continuous outer surface. The dilator and sheath together form a catheter introducer.

The present invention dilator has a design consisting of a long dilator with an approximately 1 Fr/1-1.5 cm taper, therefore obviating the need for pre-dilatation prior to sheath placement. The present invention dilator features a tip design that gradually tapers up from 4 or 5 Fr to 8 or 30 Fr, or larger, to thereby provide many advantages over existing designs. When used for applications such as central venous catheter insertion, the present invention would obviate the need to continually upsize dilators to accommodate the final catheter size. Typically, by way of example, without limiting the scope of the invention, 12 to 16 Fr dilators are used for non-tunneled and tunneled hemodialysis catheters; aortic endografts and extension limbs often utilize 12-21 Fr platforms; and 28-30 Fr dilators are often employed by urologists for laparoscopic percutaneous nephrolithotony. Dilator designs of the present invention pose no physical upper limit regarding diameter size.

Another advantage of the present invention dilator is a flexible dilator shaft portion and/or distal tapered tip portion, which provides excellent dilator tractability compared to existing stiff dilators. In one embodiment of the present invention, at least a section of the tapered tip portion is capable of bending along a guidewire with minimal physical force without kinking, to tract the curvature of a vessel, tract, or cavity. In another embodiment of the invention, at least a section of the shaft portion is capable of similarly bending along a guidewire. The present invention functions as a dilator, and tracts like a catheter over a guidewire. The present invention dilator can be used alone or with a sheath. Application of the present invention dilator system is essentially the same as any traditional dilator and vascular sheath.

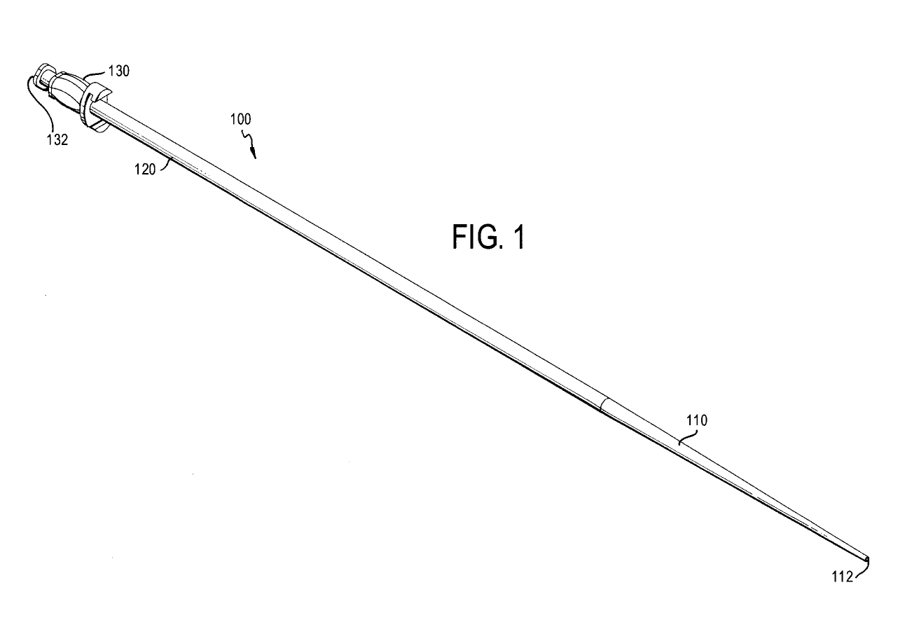

FIG. 1 illustrates one embodiment of the present invention dilator 100. The dilator 100 comprises a shaft portion 120 and a proximal hub portion 130. The shaft portion 120 transitions, at its distal end, into a long tapered tip portion 110. The exterior surface of the shaft portion 120 and the exterior surface of the long tapered tip portion 110 transition to form a smooth contour throughout, the transition between the shaft portion 120 and the long tapered tip portion 110 being free from projections, ridges or reveals. The outer diameter of the tubular shaft portion 120 defines the maximum size of the particular dilator embodiment (e.g., for a 14 Fr dilator, the outer diameter of the shaft portion of the dilator is 14 Fr).

The present invention contemplates a taper for the long tapered tip portion 110 somewhere in the range of 4 Fr/1 cm, or less. In certain embodiments, the taper for the long tapered tip portion 110 is approximately 4.0 Fr/1 cm, or 3.5 Fr/1 cm, or 3.0 Fr/1 cm, or 2.5 Fr/1 cm, or 2 Fr/1 cm, or 1.5 Fr/1 cm, or 1 Fr/1 cm, or 0.5 Fr/1 cm.

In preferred embodiments, the taper is approximately between 1 Fr/1 cm and 1 Fr/1.5 cm. In one specific embodiment, the taper for the long tapered tip portion 110 is no more than 1 Fr/1 cm (i.e., each 1 Fr change in the outer diameter of the long tapered tip portion 110 occurs over a 1 cm length of the tapered tip portion). In another specific embodiment, the long tapered tip portion 110 has a taper of between approximately 1 Fr/1 cm and 1 Fr/1.5 cm. As detailed above, taper size can vary from these preferred ranges and still be within the scope of the present invention.

The long tapered tip portion 110, the shaft portion 120, and the hub portion 130, all have a center hollow that is inter-connected to form a center channel, thereby allowing passage of a guidewire (not shown). The center channel has a distal opening 112 at the distal tip of the long tapered tip portion 110, and a proximal opening 132 at the proximal end of the hub portion 130. The inner diameter of the center channel may vary throughout the length of the dilator 100. However, at any point within the center channel of the dilator, the inner diameter of the center channel is larger than the outer diameter of the guidewire designed to be used with the dilator 100 (e.g., if the dilator 100 is to be used with a 0.035 in. guidewire, the minimal inner diameter of the center channel of the dilator is greater than 0.035 in.).

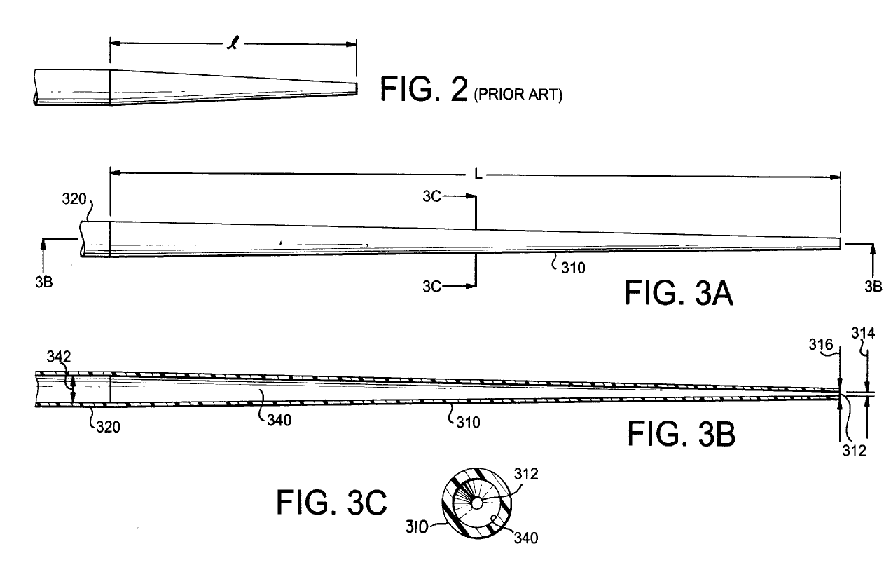

FIG. 2 illustrates a side view of the tip portion of a typical prior art dilator. Existing dilators generally have rigid, cone shaped tips. The taper length (l) of existing dilator tips are usually short, extending from 4˜5 Fr at the distal tip to a maximum diameter in about 1.5˜2.5 cm. Commercially available dilators from most manufacturers have a constant taper length (l), irrespective of the overall French size of the dilator. So, a 6 Fr dilator and a 12 Fr dilator generally have the same overall taper length (l). The shaft portions of existing dilators have a common diameter over their length, and different size dilators generally have the same shaft length.

FIG. 3A illustrates an expanded side view of a long tapered tip portion 310 of one embodiment of the present invention dilator. FIG. 3B shows a cross section taken along line 3B-3B of FIG. 3A. FIG. 3C shows a cross section taken along line 3C-3C of FIG. 3A. The long tapered tip portion 310 is connected to the distal end of the shaft portion 320 of the dilator. Distal opening 312 of the long tapered tip 310 allows the passing of a guidewire.

The length (L) of the tapered tip portion 310 is significantly longer than that of the prior art dilator shown in FIG. 2. The tapered tip portion 310 has a long, gradual taper of approximately 1 Fr/1-1.5 cm, with a fixed, total taper length (L) depending on the size of the dilator. For example, a dilator that tapers from 4 Fr (at its distal tip) to 8 Fr would have a total taper length (L) of 4˜6 cm; a dilator having a 4 Fr to 9 Fr taper would have a total taper length (L) of 5˜7.5 cm; a dilator having a 4 Fr to 10 Fr taper would have a total taper length (L) of 6˜9 cm; and a dilator having a 4 Fr to 11 Fr taper would have a total taper length (L) of 7˜10.5 cm. The outer diameter of the distal end 316 of the long tapered tip portion 110 is generally 4˜5 Fr. The center channel 340 extends through the entire length of the dilator, from the distal tip opening 312 to the proximal opening. The inner diameter of the center channel 340 of the shaft portion 342 and the inner diameter 314 of the dilator tip distal opening 312 are larger than the guidewire intended for use with the present invention dilator. In the FIGS. 3A and 3B embodiments of the present invention, a straight taper is illustrated. However, it is within the scope of the present invention to have a taper in any curved shape.

Existing dilators are generally made from a molding process. Hollow tubes are first formed from rigid plastic. One end of the hollow tube is heated and pressed in a mold. Mandrels are usually inserted in the tube to help retain the interior shape of the tip during the molding process.

The shaft portion 120 and the long tapered tip portion 110 of the present invention dilator 100 are made of flexible biocompatible polymers. A suitable polymer should have mechanical strength that retains the physical shape at the shaft portion and at the same time flexibility along the longitudinal axis. Polymers, especially thermoplastic polymers, such as polyethylene, polyurethane, and polyamides (Nylon) are some examples of possible choices. One suitable polymer is Nylon 12, which provides good mechanical strength and flexibility. The present invention dilator shaft portion 120 and the long tapered tip portion 110 can bend readily (without kinking) along the longitudinal axis of the dilator 100, but also retain mechanical strength around the circumference of the dilator shaft 120. This flexibility along the longitudinal axis of the dilator 100 provides excellent tractability.

The present invention dilator is preferably manufactured using an extrusion process. By way of illustration, without limiting the scope of the invention, the shaft portion 120 and the long tapered dilator tip 110 can be extruded using a cam controlled extruder to achieve variable taper of the dilator. The pull rate of the extrusion process can be controlled to yield different taper lengths (L) and shaft portion 120 diameters. By way of illustration, without limiting the present invention, the pull rate of the extruder is set at a high rate to form the narrowest portion of the long tapered dilator tip 110. The pull rate of the extruder then gradually slows to form the long tapered portion of the dilator tip 110. When a predetermined taper length (L) is reached, the extruder is set to a constant rate to form the shaft portion 110 of the dilator. The process can be repeated continuously. Individual sections of the long tapered dilator tip portion 110 and the shaft portion 120 are later excised from the continuous extrusion product.

Alternatively, the long tapered dilator tip portion 110 can go through an additional shaping process to form the precise size and shape of the distal tip opening 112. By way of example, without limiting the scope of the invention, the shaping process can be one of heating the distal section of the long tapered dilator tip portion 110 in a die while applying pressure. The heating of the dilator tip portion 110 can be achieved by radio frequency heating, ultrasonic heating, or direct heat. A mandrel can be placed in the center channel of the dilator tip portion 110 during the shaping process to help shape the distal tip opening 112. The hub portion 130 of the dilator is generally made by a molding process and connects to the distal end of the shaft portion 120 during final assembly. With the exception of the molded plastic hub, the present dilator is otherwise a one-piece design. It has no moving parts. It does not require additional components to accomplish its goal of subcutaneous tract dilatation. The present invention dilator is flexible along the longitudinal axis, but retains its shape and size around the circumference of the dilator shaft 120. In another embodiment, the present invention dilator can be co-extruded with two polymers arranged concentrically. The inner polymer is chosen primarily for strength. The outer polymer is chosen to be hydrophilic so that blood or other body fluid that wet and lubricate the outer surface of the dilator, make the outer surface slippery, to expedite insertion and minimize patient discomfort.

The outer dimension of the distal tip 316 of the dilator is designed according to the size of the entry point created by the puncture needle or the micro-puncture set. Generally, the outer dimension of the distal tip 316 is 4˜5 Fr. The outer dimension of the shaft portion of the dilator can be manufactured to have a variety of sizes for different applications. The size of dilators generally can vary from 8 to 16 Fr. The present invention dilator can be made to have even larger sizes without modification. Several variations of the present invention dilator, suitable for commonly used applications, can be manufactured for the ease of selection. For illustration, and without limiting the present invention, dilators that taper from: 5 Fr to 8 Fr, 5 Fr to 9 Fr, 5 Fr to 10 Fr, 5 Fr. to 11 Fr, and 5 Fr to 12 Fr can be manufactured to suit a large variety of applications. Regardless of the size of the present invention dilator, the distal tip of the dilator can always fit the initial entry hole. The desired degree of dilatation is reached by simply selecting the appropriately sized dilator and inserting the dilator to the desired depth. Since the dilator provides smooth gradual dilatation from the initial entry point to the final size, an operating physician would only need one dilator to do the job, despite the surgical need, and despite the size of the catheter or tubes required.

The inner diameter of the dilator tip opening 314 is designed to accommodate a guidewire. The inner diameter of the shaft portion 342 is usually equal to or greater than the inner diameter of the dilator tip opening 314. Commonly used guidewires can have sizes of 0.035 in and 0.038 in. For a 0.035 in guidewire, the inner diameter of the dilator tip opening 314 is chosen to be slightly larger than the guidewires. As an example, in one embodiment of the present invention, the inner diameter of the dilator tip opening 214 is chosen to be 0.041 in.

The present invention can be used alone or with a sheath. All available sheath configurations can be readily adapted for use with the present invention dilator. In cases when a catheter or other medical device is to be left in the patient for a prolonged period of time, a tear-away sheath can be used.

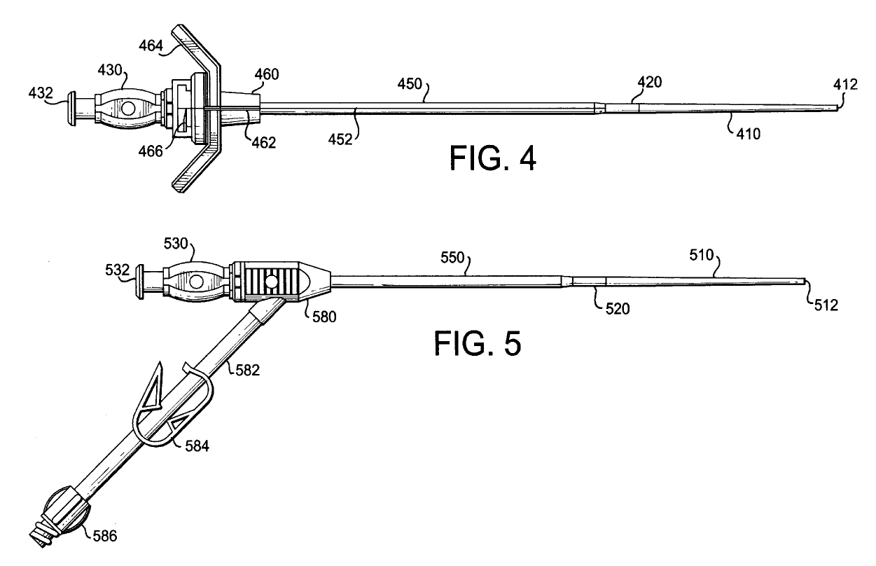

FIG. 4 shows a catheter introducer assembly with present invention dilator and a tear-away sheath. Tear-away sheaths are manufactured in a way that aids in the tearing of the sheath at two opposing points on the circumference of the sheath, thereby splitting the sheath into halves, separated longitudinally along a center line of the sheath. By splitting the sheath along its longitudinal axis as the sheath is being removed from the patient, the physician will be able to pull out the sheath in such a way that the portion removed from the patient is split, and does not interfere with encumbrances on the catheter.

The dilator of FIG. 4 comprises a distal long tapered tip portion 410, a dilator shaft portion 420, and a proximal hub portion 430. A guidewire can be passed inside the dilator through a center channel having a distal tip opening 412 and a proximal hub opening 432. The tear-away sheath comprises a distal tubular portion 450, and a proximal sheath hub portion 460. Tear seams 452 are formed parallel to the longitudinal axis of the tubular portion 450 on opposite sides of the tubular portion 450. The sheath hub 460 further comprises two halves that are weakly connected. The interface between the two sheath hub halves 462 align with the tear seams 452 of the tubular portion 450. Tabs on each of the sheath hub halves 464 provide a gripping surface for the operating physician during the insertion of the sheath and also at time of sheath removal. When the tabs are pulled away from each other, the sheath hub 460 split along the interface between the two sheath hub halves 462. The separation continues along the tear seams 452 of the tubular portion 450, and ultimately split the sheath in two.

In some applications, such as arterial intervention, a sheath with a hemostasis valve and side port is used. The hemostasis valve reduces blood leakage, and the side port provides a means to flush the sheath. FIG. 5 illustrates a catheter introducer assembly with the present invention dilator, and a sheath with a hemostasis valve and side port. The dilator comprises a distal long tapered tip portion 510, a dilator shaft portion 520, and a proximal hub portion 530. A guidewire can be passed inside the dilator through a center channel having a distal tip opening 512 and a proximal hub opening 532. The sheath comprises a distal tubular portion 550 and a proximal sheath hub portion 580. The sheath hub portion 580 further comprises an internal hemostasis valve (not shown) and a side port that connects to a side tube 582, which in turn connects to a connector 586. A clamp 484 is placed on the side tube 582 to prevent fluid flow when the side port is not in use.

There are a variety of ways to couple the dilator and sheath in a catheter introducer assembly. The present invention dilator can be modified to adopt any of these coupling means. For example, referring to FIG. 4, one embodiment of the coupling means is shown as a tongue and grove coupling configuration 466. Additional coupling means include a Luer lock connection with threaded connections.

The present invention dilator can also have markings on the exterior along the long tapered tip portion 110 and/or along the length of the dilator shaft portion 120. Graduated markings along the exterior of the long tapered tip portion can indicate the outer diameter of the long tapered tip portion 110 at that respective point along its longitudinal length. Such markings can be expressed in French sizes, or other units customarily used for dilator outer diameter by a person ordinary skilled in the art. Markings can also be made along the exterior of the dilator shaft portion 120 or on the exterior of the tubular portion of a sheath to indicate depth of the dilatation. An operating physician would then be aware of an exact degree of dilated track by visual inspection of the dilator shaft portion, dilator distal tip tapered portion, or the tubular portion of a sheath. These depth markings can be expressed in millimeters, centimeter, inches, or any other units that customarily used in the art to indicate depth/length of dilatation. These markings on the exterior of the long tapered tip portion 110 and on the exterior of the dilator shaft portion 120, or on the exterior of the tubular portion of a sheath can be made with a variety of methods. Such methods include, but are not limited to, printing, stamping, and/or molding.

The present invention dilator system helps to eliminate problems associated with existing dilators. The flexibility of present invention dilator decreases vessel trauma and diminishes risk of guidewire kinking. While functioning as a dilator, the present invention dilator tracks over a guidewire like a catheter. The elimination of repeated pre-dilatation with multiple dilators can also reduce trauma to a vessel, diminish blood loss, cause less patient discomfort, and a loss of vessel access. Since only one dilator is necessary in the present invention, thereby requiring only a single insertion operation, overall procedure time is decreased, which can be critical in cases of dissection or rupture. By decreasing the number of passes through the skin, the risk of procedure related infection is also reduced. Procedure costs are also decreased by eliminating waste.

While the present invention dilator can be used for vessel access, there are many other possible uses. By way of illustration, without limiting the scope of the invention, the dilator of the present invention can replace existing dilators for recurrent/refractory ascites catheters, percutaneous nephrostomy tubes, biliary tubes, abscess drains, thoracentesis and paracentesis drains, etc.

These and other advantages of the present invention will be apparent to those skilled in the art from the foregoing specification. Accordingly, it will be recognized by those skilled in the art that changes or modifications may be made to the above-described embodiments without departing from the broad inventive concepts of the invention. It should therefore be understood that this invention is not limited to the particular embodiments described herein, but is intended to include all changes and modifications that are within the scope and spirit of the invention.

We claim:

1. A dilator assembly for the dilatation of vessel, tract, or cavity, comprising:
a dilator, having a proximal hub portion, a shaft portion, and a flexible distal tapered tip portion;
wherein the shaft portion and the distal tapered tip portion have an exterior surface;
wherein the exterior surface of the shaft portion smoothly transitions to the exterior surface of the flexible distal tapered tip portion;
wherein the hub portion, the shaft portion, and the flexible distal tapered tip portion have a continuous hollow center that forms a guidewire passage.

2. The dilator assembly as in claim 1, wherein an outer diameter of a distal end of the flexible distal tapered tip portion is about French size 4 to French size 5.

3. The dilator assembly as in claim 1, wherein an outer diameter of the shaft portion is about French size to French size 30.

4. The dilator assembly as in claim 1, wherein the flexible distal tapered tip portion has a taper of no greater than 4 Fr per 1 cm.

5. The dilator assembly as in claim 1, wherein the flexible distal tapered tip portion is tapered between approximately 4 Fr per 1 cm and 1 Fr per 2 cm.

6. The dilator assembly as in claim 1, wherein the flexible distal tapered tip portion is tapered between approximately 2 Fr per 1 cm and 1 Fr per 2 cm.

7. The dilator assembly as in claim 1, wherein the flexible distal tapered tip portion is tapered between approximately 1 Fr per 1 cm and 1 Fr per 1.5 cm.

8. The dilator assembly as in claim 1, wherein the flexible distal tapered tip portion and the shaft portion are made of a flexible thermoplastic polymer.

9. The dilator assembly as in claim 1, wherein the flexible distal tapered tip portion and the shaft portion are made of Nylon 12.

10. The dilator assembly as in claim 1, wherein the flexible distal tapered tip portion and the shaft portion are made by an extrusion process.

11. The dilator assembly as in claim 1, wherein the exterior surface of the flexible distal tapered tip portion has at least one marking indicating a value of an outer diameter of the flexible distal tapered tip portion at one or more locations along a longitudinal length of the flexible distal tapered tip portion.

12. The dilator assembly as in claim 1, wherein the exterior surface of the shaft portion has at least one marking indicating a value of an outer diameter of the shaft portion at one or more locations along a longitudinal length of the shaft portion.

13. The dilator assembly as in claim 1, further comprising:
a sheath, having a tubular portion and a sheath hub portion.

14. The dilator assembly as in claim 13, wherein the sheath is a tear-away sheath.

15. The dilator assembly as in claim 13, wherein the sheath comprises a hemostasis valve within the sheath hub portion and a side port.

16. The dilator assembly as in claim 13, wherein the dilator further comprises a coupling means at a distal end of the dilator hub portion that couples to the sheath hub portion.

17. The dilator assembly as in claim 13, wherein the tubular portion has an exterior surface, and the exterior surface of the tubular portion has at least one marking indicating a respective length from a distal end of the tubular portion.

18. A method of making a tapered tip dilator comprising the steps of:
forming a shaft portion and a tapered tip portion with a hollow center channel using a variable speed extrusion process;
separating an individual section of a shaft portion and a tapered tip portion, wherein the center channel extends to a proximal opening at a proximal end of the shaft portion and to a distal opening at a distal end of the tapered tip portion; and
shaping the distal tip opening of the tapered tip portion.

19. The method of claim 18 wherein shaping the distal tip opening comprises the steps of:
heating a section of the long tapered tip portion; and
compressing the long tapered tip portion in a die, thereby forming the distal tip opening.

20. The method of claim 18, further comprising the steps of:
forming a dilator hub having a distal end, a proximal end, and a hollow center channel; and
connecting the distal end of the dilator hub to the proximal end of the shaft portion, whereby the dilator hub, the shaft portion, and the flexible distal tapered portion have a continuous center channel forming a guide wire passage.
